(19) United States
(12) Patent Application Publication
(10) Pub. No.: US 2024/0048595 A1
(43) Pub. Date: Feb. 8, 2024

(54) INTEGRATED HEALTHCARE EXCHANGE

(71) Applicant: TeleTracking Technologies, Inc., Pittsburgh, PA (US)

(72) Inventors: Michelle Suzanne Skinner, Gallatin Gateway, MT (US); Elizabeth Ann Anderson, Crescent, PA (US); Raymond M. Powell, JR., Sewickley, PA (US)

(21) Appl. No.: 18/363,476

(22) Filed: Aug. 1, 2023

Related U.S. Application Data
(60) Provisional application No. 63/394,325, filed on Aug. 2, 2022.

(51) Int. Cl. H04L 9/40; G16H 40/20

(52) U.S. Cl. CPC H04L 63/20; G16H 40/20; H04L 63/0428; G16H 10/60

(57) ABSTRACT

One embodiment provides a method, the method including: providing a healthcare system exchange facilitating communicative connection of at least two healthcare systems; receiving, at the healthcare system exchange, and storing, within a database of the healthcare system exchange, a set of communication rules corresponding to communications occurring between the at least two healthcare systems, the set of communication rules defining information that can be shared between the at least two healthcare systems; receiving, at the healthcare system exchange from a first of the at least two healthcare systems, a request for access to a resource of at least a second of the at least two healthcare systems; and transmitting, from the healthcare system exchange to the at least a second of the at least two healthcare systems, the request, wherein the transmitting includes ensuring the request conforms with the set of communication rules. Other aspects are claimed and described.

### CROSS REFERENCE TO RELATED APPLICATION

This application claims priority to U.S. Patent Application Ser. No. 63/394,325, filed on Aug. 2, 2022, and entitled “INTEGRATED HEALTHCARE EXCHANGE,” the contents of which are incorporated by reference herein.

### BACKGROUND

Healthcare systems are a critical part of a community's infrastructure. The healthcare system provides services necessary for ensuring the health and safety of a community. Healthcare systems can include many different healthcare facilities, for example, emergency departments, acute-care facilities, post-recovery facilities, long-term care facilities, surgical facilities, general care facilities, urgent care facilities, clinics, ambulatory surgery centers, and the like. Each of these facilities provides different healthcare services, each assisting in ensuring the health, well-being, and safety of the community the healthcare system serves.

### BRIEF SUMMARY

In summary, one aspect provides a method, the method including: providing a healthcare system exchange, wherein the healthcare system exchange facilitates communicative connection of at least two healthcare systems; receiving, at the healthcare system exchange, and storing, within a database of the healthcare system exchange, a set of communication rules corresponding to communications occurring between the at least two healthcare systems, the set of communication rules defining information that can be shared between the at least two healthcare systems; receiving, at the healthcare system exchange from a first of the at least two healthcare systems, a request for access to a resource of at least a second of the at least two healthcare systems; and transmitting, from the healthcare system exchange to the at least a second of the at least two healthcare systems, the request, wherein the transmitting includes ensuring the request conforms with the set of communication rules.

Another aspect provides a system, the system including: a database, wherein the database includes a set of communication rules corresponding to communications occurring between at least two healthcare systems, the set of communication rules defining information that can be shared between the at least two healthcare systems; at least one network device facilitating communicative connection between a healthcare system exchange and the at least two healthcare systems; a processor operatively coupled to the database and the at least one network device; a memory device that stores instructions that, when executed by the processor, causes the information handling device to: provide the healthcare system exchange, wherein the healthcare system exchange facilitates communicative connection of at least two healthcare systems based upon the set of communication rules corresponding to the at least two healthcare systems; receive, at the healthcare system exchange from a first of the at least two healthcare systems, a request for access to a resource of at least a second of the at least two healthcare systems; and transmit, from the healthcare system exchange to the at least a second of the at least two healthcare systems, the request, wherein the transmitting includes ensuring the request conforms with the set of communication rules.

A further aspect provides a product, the product including: a computer-readable storage device that stores executable code that, when executed by a processor, causes the product to: provide a healthcare system exchange, wherein the healthcare system exchange facilitates communicative connection of at least two healthcare systems; receive, at the healthcare system exchange, and storing, within a database of the healthcare system exchange, a set of communication rules corresponding to communications occurring between the at least two healthcare systems, the set of communication rules defining information that can be shared between the at least two healthcare systems; receive, at the healthcare system exchange from a first of the at least two healthcare systems, a request for access to a resource of at least a second of the at least two healthcare systems; and transmit, from the healthcare system exchange to the at least a second of the at least two healthcare systems, the request, wherein the transmitting includes ensuring the request conforms with the set of communication rules.

A further aspect provides a method, the method including: transmitting, from a first of at least two healthcare systems, a request for access to a resource of at least a second of the at least two healthcare systems, wherein the at least two healthcare systems are communicatively connected via a healthcare system exchange, wherein the healthcare system exchange stores a set of communication rules corresponding to communications occurring between the at least two healthcare systems, the set of communication rules defining information that can be shared between the at least two healthcare systems; the transmission of the request being facilitated via the healthcare system exchange, wherein the healthcare system exchange conforms the request with the set of communication rules; and receiving, at the first of the at least two healthcare systems via the healthcare system exchange, a status responsive to the request, wherein the transmission of the status is facilitated via the healthcare system exchange and wherein the healthcare system exchange conforms the status with the set of communication rules.

A further aspect provides a method, the method including: receiving, at a second of at least two healthcare systems and from a first of at least two healthcare systems, a request for access to a resource of the least a second of the at least two healthcare systems, wherein the at least two healthcare systems are communicatively connected via a healthcare system exchange, wherein the healthcare system exchange stores a set of communication rules corresponding to communications occurring between the at least two healthcare systems, the set of communication rules defining information that can be shared between the at least two healthcare systems; the transmission of the request being facilitated via the healthcare system exchange, wherein the healthcare system exchange conforms the request with the set of communication rules; and transmitting, from the second of the at least two healthcare systems to the first of the at least two healthcare systems via the healthcare system exchange, a status responsive to the request, wherein the transmission of the status is facilitated via the healthcare system exchange and wherein the healthcare system exchange conforms the status with the set of communication rules.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example method of providing a healthcare system exchange facilitating communicative connection of at least two healthcare systems.

FIG. 2 illustrates an example communication diagram of healthcare systems with the healthcare system exchange.

FIG. 3 illustrates an example of device circuitry.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

Healthcare facility refers to a single department within a healthcare facility or a single healthcare facility which generally means a healthcare service provider within a single building or set of buildings on a campus.

Healthcare system refers to a system of healthcare facilities, which may be a single facility, that are serviced by the same healthcare service provider and may be further defined by the same healthcare service provider in a geographical area. It should be noted that the healthcare facilities within may or may not be connected, meaning the healthcare facilities within the healthcare system may or may not be able to access systems of or electronically communicate with other facilities within the healthcare system.

Affiliated healthcare systems refer to healthcare systems serviced by different healthcare service providers, but that have an agreement that officially connects the two healthcare systems, meaning the healthcare systems have an official relationship to each other. Affiliated healthcare systems may also include affiliated ancillary entities. Affiliated healthcare systems are generally unable to access systems of or electronically communicate with other facilities within the affiliated healthcare systems.

Non-affiliated healthcare systems refer to healthcare systems serviced by different healthcare service providers and that do not have an agreement that officially connects the two healthcare systems. Non-affiliated healthcare systems may also include non-affiliated ancillary entities. Non-affiliated healthcare systems are generally unable to access systems of or electronically communicate with other facilities within the non-affiliated healthcare systems.

Each healthcare facility in a healthcare system has its own system for tracking resources and patients within the facility. For example, the facility may have a one or more systems that track patients, healthcare staff, healthcare equipment and objects, and/or the like. These systems generally are able to communicate between each other within the healthcare facility. However, communications that need to go outside a healthcare facility are more difficult to support. This is because the healthcare industry is subject to strict privacy and security requirements with respect to medical information. Thus, transmitting information outside of a healthcare facility or receiving information from outside the healthcare facility requires additional processes and procedures to ensure the security and privacy of the transmitted information, which typically results in manual transmission of information, for example, using facsimiles, telephone calls, or couriers.

Some systems have been implemented that allow for the transmission of information to healthcare facilities within a healthcare system. These systems are designed to ensure the security and privacy of information shared within the healthcare system. Since the healthcare system is interconnected, meaning serviced by the same healthcare service provider, the systems within with the healthcare system are similar. Since the systems are similar, the communication protocols and other communication transmission techniques are known by each of the systems. Additionally, since the systems are serviced by the same healthcare provider, specific communication channels and protocols can be provided to ensure that communications transmitted using the communication channels and protocols can be kept secure and private.

However, not all healthcare systems are interconnected, even if serviced by the same healthcare provider, thereby increasing the difficulty in providing specific communication channels and protocols, if possible at all. Additionally, affiliated and non-affiliated healthcare systems may not have the same systems as each other, thereby making transmission of secure and private communications more difficult. Additionally, since the affiliated and non-affiliated healthcare systems are not serviced by the same healthcare provider, creating specific communication channels or protocols is difficult, particularly for disparate systems.

Thus, information that needs to be conveyed between healthcare facilities, whether in the same healthcare system, within affiliated healthcare systems, or within non-affiliated healthcare systems is generally performed manually where a person from one facility calls or faxes a person from another facility to gain the desired information. Accordingly, the information has to be conveyed outside any tracking systems of a healthcare facility or systems. It is then necessary for the information to be manually put into the tracking systems of the healthcare facility or system.

Accordingly, an embodiment provides a system and method for providing a healthcare system exchange facilitating communicative connection of at least two healthcare systems. A healthcare system exchange is provided that facilitates communicative connection of at least two healthcare systems, which as previously mentioned may include a single healthcare facility. Instead of requiring the healthcare systems to create and provide communication channels and protocols between the two healthcare systems, the healthcare system exchange provides secure and private communication between the healthcare systems. Thus, the healthcare systems simply connect to the healthcare system exchange and any communications that a healthcare facility wants to transmit to another healthcare facility are transmitted through the healthcare system exchange which ensures the security and privacy of the transmitted and received communications. Accordingly, the healthcare system exchange can connect any types of healthcare systems including facilities within the same healthcare system, affiliated healthcare systems, and non-affiliated healthcare systems.

Since healthcare systems may not want every system of the healthcare system to be accessible to other healthcare systems, the healthcare system exchange receives and stores a set of communication rules that correspond to communications occurring between two healthcare systems or groups of healthcare systems. These set of rules define information that can be shared between the healthcare systems covered by the set of rules. Thus, information that healthcare systems do not want shared with other healthcare systems is kept from the other healthcare systems. Each set or group of connected healthcare systems can have its own set of communication rules that the healthcare system exchange will ensure is met. In other words, the healthcare system exchange can vary communication protocols between healthcare systems based upon the set of communication rules corresponding to the healthcare systems.

When a first of the at least two healthcare systems wants to make a request for a resource of a second of the at least two healthcare systems, the request is received at the exchange. The exchange then transmits the request to the second of the at least two healthcare systems. However, the exchange is not simply a forwarder of the request. Rather, the exchange ensures the request conforms with the set of communication rules. Thus, the exchange may augment the request so that it conforms with the set of communication rules. Similarly, when a response to the request is transmitted back to the first of the at least two healthcare systems, the exchange ensures the response conforms with the communication rules. Thus, the exchange not only ensures that the communications between healthcare systems is transmitted securely and privately, but also ensures that only information that has been agreed upon by the healthcare systems is revealed to the other of the healthcare systems.

It should be noted that the first and second healthcare systems may change based upon the role of the healthcare system. For example, the first healthcare system may make a request to transfer a patient to a second healthcare system. When the patient is ready to be moved back to the first healthcare system, the second healthcare system may make a request to transfer the patient back to the first healthcare system. Thus, in this example, the second healthcare system would be the first healthcare system in the communication exchange and the first healthcare system would now be the second healthcare system. In other words, the terms first and second are not intended to designate particular healthcare systems, but rather roles of the healthcare systems, with the first healthcare system being the healthcare system providing the request and the second healthcare system being the healthcare system receiving the request.

Providing a healthcare exchange that allows communication among healthcare systems has unique challenges. Due to government regulations, privacy laws, and other rules and procedures, transmission of medical information, patient information, healthcare system information, and other information associated with healthcare (collectively referred to as “healthcare information”) is difficult in that many different systems must be in place to ensure the security and privacy of such information. Thus, providing a healthcare exchange is uniquely different than providing other types of exchanges such as selling exchanges, communication exchanges, and/or the like. Additionally, if the healthcare information is to be transmitted outside a single healthcare system to another healthcare system, even an affiliated healthcare system, requires extra procedures and protocols to ensure the security and privacy of the healthcare information. To transmit the healthcare information to a non-affiliated healthcare system, requires even more procedures and protocols. Accordingly, the ability to provide such a exchange while ensuring security and privacy of the information that may be transmitted is a great technical advancement in the healthcare field. Such a healthcare exchange is provided in the described systems and methods.

The illustrated example embodiments will be best understood by reference to the figures. The following description is intended only by way of example, and simply illustrates certain example embodiments.

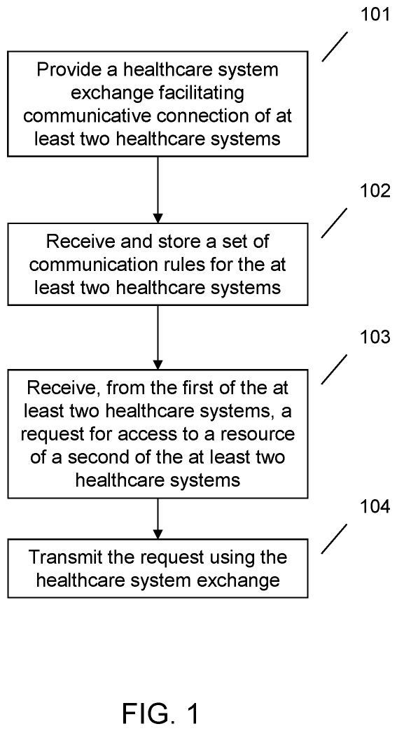

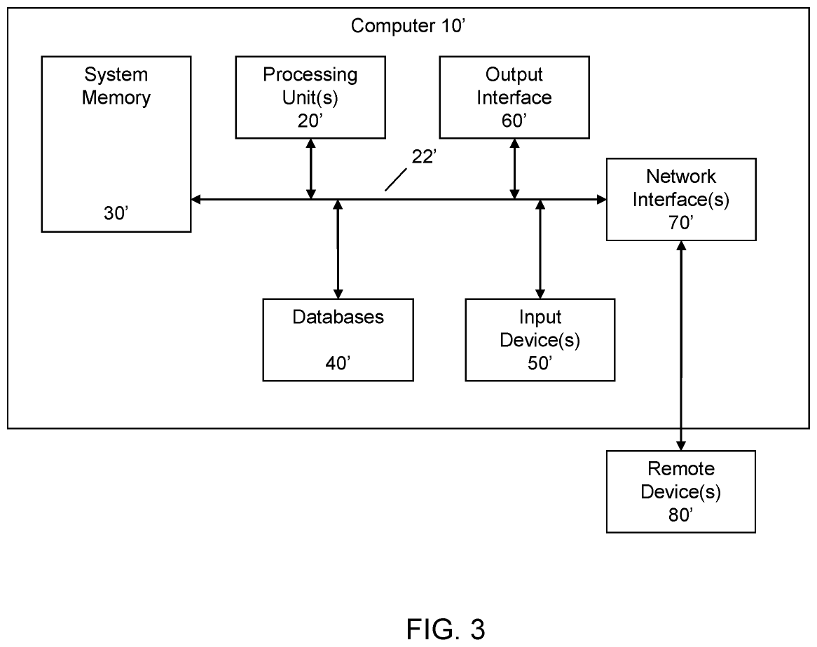

FIG. 1 illustrates an example method for providing a healthcare system exchange facilitating communicative connection of at least two healthcare systems. The method may be implemented on a system which includes a processor, memory device, output devices (e.g., display device, printer, etc.), input devices (e.g., keyboard, touch screen, mouse, microphones, sensors, biometric scanners, etc.), image capture devices, and/or other components, for example, those discussed in connection with FIG. 3. While the system may include known hardware and software components and/or hardware and software components developed in the future, the system itself is specifically programmed to perform the functions as described herein to provide a healthcare system exchange that facilitates communicative connection between healthcare systems. Additionally, the healthcare system exchange includes modules and features that are unique to the described system.

At 101 a healthcare system exchange is provided. The healthcare system exchange facilitates communicative connection of at least two healthcare systems. In other words, the healthcare system exchange provides a mechanism to communicate between two or more healthcare systems. Transmissions from one healthcare system to another healthcare system are facilitated through the healthcare exchange to ensure the privacy and security of the communications. Additionally, the healthcare system exchange ensures that only information agreed upon by the healthcare systems is shared with other healthcare systems.

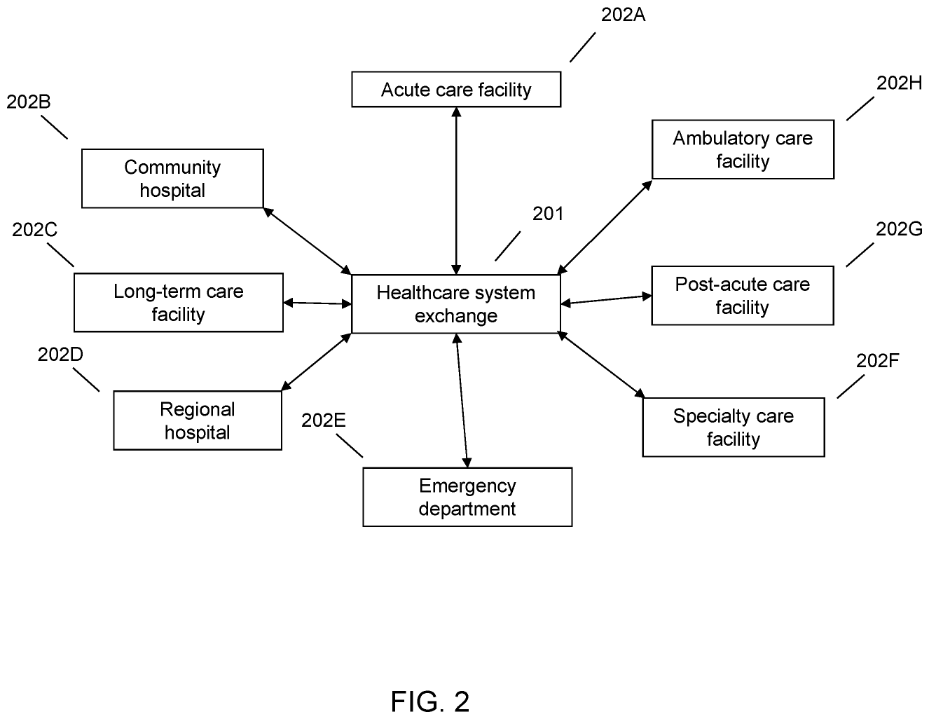

An example communication diagram is illustrated in FIG. 2. The healthcare system exchange 201 can facilitate communication between different healthcare systems such as acute-care facilities 202A, community hospitals 202B, long-term care facilities 202C, regional hospitals 202D, emergency departments 202E, specialty-care facilities 202F, post-acute care facilities 202G, ambulatory care facilities 202H, and/or the like. The healthcare system exchange 201 can facilitate communication between any of the healthcare systems 202A-202H that have a set of communication rules. For example, the acute-care facility 202A can communicate with the emergency department 202E through the healthcare system exchange 201.

It should be noted that the healthcare systems included in this figure are merely examples and other healthcare systems may be included. Additionally, the number of healthcare systems may vary. Additionally, since the exchange facilitates communication with many different healthcare systems, many different instances of a single type of healthcare system may be connected to the exchange. Each of the healthcare systems identified may also include other sub-healthcare systems which may individually communicate with the exchange.

Referring back to FIG. 1, the healthcare system exchange, also referred to as the healthcare exchange or exchange, may include or have access to network devices. The network devices may facilitate communication between the healthcare system exchange and another device, component, facility, healthcare system, and/or the like. For example, the exchange may utilize a network device to communicate with the healthcare systems that are providing and receiving transmissions.

It should be noted that two healthcare systems are discussed herein. However, this is simply for ease of readability. Rather, multiple healthcare systems may be interconnected by the healthcare exchange in healthcare system groups. This means that a healthcare system could transmit a request to multiple of the other healthcare systems within the group.

The exchange may have an associated graphical user interface. The exchange may be a background application that does not have an associated display or monitor. Rather, the exchange, when accessed by a user will provide instructions to generate and display a graphical user interface on a display device utilized by the user. Thus, the exchange may not have its own display device, but does have the ability to generate and display a graphical user interface. The graphical user interface is updated and managed based upon instructions provided by the exchange. In other words, the exchange generates and transmits instructions to create and update the graphical user interface.

The graphical user interface may allow a user to generate a request to be sent to another healthcare system. The graphical user interface may also display a status or information associated with the request, for example, when the request was submitted, the current status of the request, a user who submitted the request, a department, facility, or healthcare system that generated the request or that the request was sent to, a patient, procedure, healthcare object, or the like, that is the resource associated with the request, and/or the like.

The graphical user interface may include one or more graphical elements. The graphical elements may include user input fields that facilitate the generation of a request. The user input fields may also allow a user to provide input to view pending requests, historical requests, statistics of requests (e.g., number or percentage of approved requests, departments generating requests, most frequent requests, most frequent request types, etc.), and/or the like. Input fields may include radial selector buttons, pull-down menus, free-form fields, structured input fields, selection boxes, and/or any other type of input field. The graphical elements may include icons that a user can interact with.

Some icons may also be display icons that display information. The display icons may be static icons that have static visual elements. The display icons may be dynamic icons that have dynamic visual elements that change based upon an update in information displayed on the icon, periodically, based upon a condition being met, and/or the like. For example, the graphical element may be a display icon that displays the status of a request. As the status is updated, the graphical element is updated to reflect the new status. In other words, the graphical element is iteratively updated based upon changes to the status of the request.

Different graphical user interfaces may be provided for different roles within the exchange. A graphical user interface for generating and transmitting a request for a resource may be generated for a first healthcare system making a request. A second, different graphical user interface may be generated, based upon instructions provided by the exchange, for a second healthcare system receiving a request for a resource. This graphical user interface may also include graphical elements as previously described. Some of these graphical elements may correspond to be pending or historical requests received by the healthcare system. Statistics and other information may also be provided in the graphical user interface as with the previously described graphical user interface.

This graphical user interface, however, may also include graphical elements allowing for the provision of status updates for pending requests. For example, the graphical user interface may include icons that allow a user to update the status of the request, approve the request, deny the request, propose modifications to the request, and/or the like. Additionally, graphical user interfaces may be modified for different healthcare systems based upon needs or desires of the healthcare system. Thus, the exchange can provide graphical user interfaces that are directed to information important to the healthcare system accessing the graphical user interface.

A user may access the exchange utilizing one or more of a variety of techniques. How the exchange is accessed may be dependent on how the exchange is interfaced with the healthcare systems. One interface technique includes the exchange being installed or located on a system, device, and/or component of the healthcare system. Another interface technique includes the exchange being installed or location on a remote location (e.g., cloud location, remote network location, etc.). When the healthcare system accesses the exchange, the healthcare system accesses the remote location to access the exchange. It should be noted that exactly how the healthcare system accesses the exchange may be accomplished using a variety of techniques and may be performed as a background process that does not require the user to perform any active steps to utilize the exchange, thereby appearing as a seamless connection between the healthcare system and the exchange and/or other healthcare system component, device, application, and/or system.

At 102, the healthcare system exchange receives and stores a set of communication rules corresponding to communications occurring between at least two healthcare systems. The set of communication rules may be stored in a database of or accessible by the healthcare system exchange. The set of communication rules define information that can be shared between the two healthcare systems, or healthcare systems within a group. It should be noted that even healthcare systems within a group may have their own communication rules. For example, all of the healthcare systems within a group may be subject to a first overarching set of communication rules. However, some of the healthcare systems within the group may have additional communication rules that apply when those healthcare systems are communicating with each other. Thus, these individual sets of communication rules may add to or override parts of the overarching set of communication rules.

Additionally, the exchange can store multiple sets of communication rules for different healthcare systems and vary communication protocols between different healthcare systems so that communications occurring between two healthcare systems are in conformance with the communication rules corresponding to those two healthcare systems. In other words, the exchange has the ability to provide unique communication structures and protocols to each set or group of healthcare systems in order to ensure that the communication rules of the set or group of healthcare systems is fulfilled, even though those communication rules are different than communication rules for other healthcare system sets or groups.

The set of communication rules may also apply to communications between healthcare systems and a central healthcare system, but may not apply between the healthcare systems. In other words, the set of communication rules may identify information that may be shared between each of the healthcare systems and the central healthcare system. However, the peripheral healthcare systems may not have a set of communication rules that allow them to communicate with each other. In this case, the exchange would only facilitate communications that include the central healthcare systems and would prevent communications between only peripheral healthcare systems.

When making an agreement to allow healthcare systems to access other healthcare systems, the systems may make an agreement regarding what information will be shared. This agreement makes up the set of communication rules. The exchange ensures that communications occurring between the healthcare systems conforms to the set of communication rules. The communication rules can define any type of information that may be shared. However, some non-limiting examples include, a status of a patient, a status of a facility, a status of a healthcare provider, a status of a healthcare staff member, a status of a piece of healthcare equipment, and/or the like. Thus, the communication rules may be as broad as allowing the other healthcare system to effectively access the tracking systems of the healthcare system to see if a provider, facility, procedure, bed within a department, and/or the like is available or when it might be available or it might only allow the healthcare system to receive a status of a request and nothing else.

At 103 the healthcare system exchange receives a request from a first of the two healthcare systems. The request is to be transmitted to a second of the two healthcare systems. As previously noted, the request may actually be transmitted to more than one healthcare system. Also as previously noted, the request may be effectuated at the first healthcare system via accessing the graphical user interface associated with the healthcare exchange and providing input to generate the request. The request may be a request for access to a resource of the second of the two healthcare systems. For example, the request may be to schedule a particular healthcare provider, to schedule a particular procedure, to transfer a patient of the first healthcare system to a particular department of the second healthcare system, to schedule a particular piece of healthcare equipment, to transfer a piece of healthcare equipment to the first healthcare system, to access a particular facility of the second healthcare system, and/or any other request for a resource. Thus, the resource may be a healthcare provider, a bed, a healthcare support staff, a procedure, a piece of healthcare equipment, a particular facility, and/or the like.

Another technique for effectuating the request may be an automatic technique. As previously noted, each healthcare system generally has its own tracking systems. These tracking systems may allow not only a current tracking of objects or people within a healthcare facility, but may also provide an indication of future tracking of objects or people within the healthcare facility. In other words, the tracking systems may allow for predicting a future expected location of an object or person, a future resource need of an object or person, and/or the like. Leveraging this prediction, the request may be generated automatically to fulfill the predicted location, resource need, and/or the like. For example, if the tracking system determines that a patient will need a procedure that is not available at the healthcare system, the tracking system may automatically generate a request for the procedure at the second healthcare system and transmit it to the exchange. Thus, in this example, the request is generated automatically based upon a status of the patient within the first healthcare system.

Upon receiving the request, the exchange transmits the request to the second of the healthcare systems at 104. However, before transmitting the request, the exchange ensures that the request conforms with the set of communication rules that apply to communications between the first healthcare system and the second healthcare system. Ensuring the request conforms to the set of communication rules may include the exchange accessing the set of communication rules that applies to communications between the first healthcare system and the second healthcare systems. Otherwise, the communication channels corresponding to communications between the first and second healthcare system may be programmed with the communication rules meaning the communication rules do not have to be accessed.

Based upon the communication rules, the exchange augments the communication, if needed, to conform with the communication rules. Augmenting the communication may include removing information included in the communication, adding information to the communication, changing information included in the communication, and/or the like. It should be noted that, depending on the communication rules, the communication may not have to be augmented. In this case, however, the exchange still ensures that the communication conforms with the communication rules even if the exchange does not ultimately augment the communication. Once the exchange has ensured that the communication conforms to the communication rules, the exchange transmits the request to the second healthcare system.

In response to the request, the second healthcare system may provide a status update related to the request. This status update may be transmitted to and received by the exchange, much like the request was transmitted to and received by the exchange. The status update may be provided automatically by the second healthcare system or may be provided manually by a user within the second healthcare system. In the event that the status update is provided manually, the user may access a graphical user interface of the exchange that allows for providing updates to the status of a request.

Once the status update is received at the exchange, the exchange may transmit the status update to the first or requesting healthcare system. However, similar to the transmission of the request, the transmission of the status update is ensured to conform with the set of communication rules applying to the first and second healthcare systems. In other words, the exchange verifies if the communication conforms to the set of communication rules. In the event that the status update does not conform to the set of communication rules, the exchange augments the communication to ensure that the communication transmitted to the requesting healthcare system conforms with the communication rules.

It should be noted that not just status updates may be transmitted by the second healthcare system or receiving healthcare system. Rather, the receiving healthcare system may transmit other communications, for example, a responsive request, a note from a healthcare provider associated with the request, a modification to the request, temporary access to a system of the second healthcare system that facilitates the request, and/or the like. These communications will be treated the same as the status update, meaning the exchange will ensure that these communications conform with the set of communication rules.

As an overall example, a community hospital has a patient who needs a magnetic resonance imaging (MRI) procedure performed. However, the community hospital does not have the facilities to provide a service. A larger healthcare system within the same geographical area does have a facility that provides MRI procedures. The community hospital accesses the graphical user interface associated with the healthcare system exchange and makes a request to the larger healthcare system for the MM procedure. The healthcare exchange identifies the set of communication rules that correspond to this community hospital and the larger healthcare system. For this example, the simplistic rules indicate that patient information cannot be conveyed within requests unless the request is approved and responses to requests only include a status of the request.

The healthcare exchange transmits the request to the larger healthcare system while ensuring the privacy and security of the communication and in view of the communication rules for the community hospital and the larger healthcare system. Based on the rules, the exchange removes all patient information from the request and then transmits the augmented request to the larger healthcare system. The larger healthcare system acts on the request and, as progress is made on the request, a status is transmitted back to the community hospital from the larger healthcare system through the exchange. Since the communication rules indicate that only a status of the request is provided, the exchange augments the status updates received from the larger healthcare system to conform to the communication rules. The exchange generates instructions to iteratively update a graphical element corresponding to the request status with the updated status as it changes.

In this example, one of the healthcare systems, the community hospital, was request access to a resource of the larger healthcare system because the community hospital does not provide such a service. However, requests do not have to be made based upon whether the requesting healthcare system could themselves provide the resource. Rather, requests can be generated and transmitted for any reason. Additionally, the above example indicated that the healthcare system is in the same geographical area as the community hospital. This is not necessary as the healthcare exchange can be deployed nationwide or even worldwide. Thus, a resource could be requested from a healthcare system in another country and not just in the same geographical area.

Accordingly, the healthcare system exchange acts as a hub that allows communication between healthcare systems, whether within the same healthcare system, affiliated healthcare systems, and/or non-affiliated healthcare systems, while still ensuring the privacy and security of the healthcare information transmitted within the communications. Additionally, the exchange can provide communication capabilities between any types of healthcare systems, even non-affiliated healthcare systems, which allows information to be conveyed between the healthcare systems.

While various other circuits, circuitry or components may be utilized in information handling devices, with a computer, server, client device or the like, an example device that may be used in implementing one or more embodiments includes a computing device in the form of a computer 10′. This example device may be a server used in one of the systems in a hospital network, or one of the remote computers connected to the hospital network. Components of computer 10′ may include, but are not limited to, a processing unit 20′, a system memory 30′, and a system bus 22′ that couples various system components including the system memory 30′ to the processing unit 20′. Computer 10′ may include or have access to a variety of computer readable media, including databases. The system memory 30′ may include non-signal computer readable storage media, for example in the form of volatile and/or nonvolatile memory such as read only memory (ROM) and/or random access memory (RAM). By way of example, and not limitation, system memory 30′ may also include an operating system, application programs, other program modules, and program data.

A user can interface with (for example, enter commands and information) the computer 10′ through input devices 50′. A monitor or other type of device can also be connected to the system bus 22′ via an interface, such as an output interface 60′. The computer may include a database 40′. In addition to a monitor, computers may also include other peripheral output devices. The computer 10′ may operate in a networked or distributed environment using logical connections to one or more other remote device(s) 80′ such as other computers. The logical connections may include network interface(s) 70′ to a network, such as a local area network (LAN), a wide area network (WAN), and/or a global computer network, but may also include other networks/buses.

Information handling device circuitry, as for example outlined in FIG. 3, may be used in client devices such as a personal desktop computer, a laptop computer, or smaller devices such as a tablet or a smart phone. In the latter cases, i.e., for a tablet computer and a smart phone, the circuitry outlined in FIG. 3 may be adapted to a system on chip type circuitry. The device, irrespective of the circuitry provided, may provide and receive data to/from another device, e.g., a server or system that coordinates with various other systems. As will be appreciated by one having ordinary skill in the art, other circuitry or additional circuitry from that outlined in the example of FIG. 3 may be employed in various electronic devices that are used in whole or in part to implement the systems, methods and products of the various embodiments described herein.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device that are executed by a processor. A storage device may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a storage device is not a signal and “non-transitory” includes all media except signal media.

Program code embodied on a storage medium may be transmitted using any appropriate medium, including but not limited to wireless, wireline, optical fiber cable, RF, et cetera, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and program products according to various example embodiments. It will be understood that the actions and functionality may be implemented at least in part by program instructions. These program instructions may be provided to a processor of a device, a special purpose information handling device, or other programmable data processing device to produce a machine, such that the instructions, which execute via a processor of the device implement the functions/acts specified.

It is worth noting that while specific blocks are used in the figures, and a particular ordering of blocks has been illustrated, these are non-limiting examples. In certain contexts, two or more blocks may be combined, a block may be split into two or more blocks, or certain blocks may be re-ordered or re-organized as appropriate, as the explicit illustrated examples are used only for descriptive purposes and are not to be construed as limiting.

As used herein, the singular “a” and “an” may be construed as including the plural “one or more” unless clearly indicated otherwise.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method, the method comprising:
providing a healthcare system exchange, wherein the healthcare system exchange facilitates communicative connection of at least two healthcare systems;
receiving, at the healthcare system exchange, and storing, within a database of the healthcare system exchange, a set of communication rules corresponding to communications occurring between the at least two healthcare systems, the set of communication rules defining information that can be shared between the at least two healthcare systems;
receiving, at the healthcare system exchange from a first of the at least two healthcare systems, a request for access to a resource of at least a second of the at least two healthcare systems; and
transmitting, from the healthcare system exchange to the at least a second of the at least two healthcare systems, the request, wherein the transmitting comprises ensuring the request conforms with the set of communication rules.

2. The method of claim 1, further comprising providing a graphical user interface associated with the healthcare system exchange.

3. The method of claim 2, wherein the graphical user interface displays at least one user input field for generating the request for access.

4. The method of claim 2, wherein the graphical user interface is provided within a system of at least one of the at least two healthcare systems and is updated and managed based upon instructions provided by the healthcare system exchange.

5. The method of claim 1, wherein the at least two healthcare systems comprise non-affiliated healthcare systems.

6. The method of claim 1, further comprising receiving, at the healthcare system exchange from the at least a second of the at least two healthcare systems, a status update related to the request; and
transmitting, from the healthcare system exchange to the first of the at least two healthcare systems, the status, wherein the transmitting comprises ensuring the status conforms with the set of communication rules.

7. The method of claim 6, wherein the status is displayed as at least one graphical element within a graphical user interface associated with the healthcare system exchange; and
wherein the at least one graphical element is iteratively updated based upon changes to the status as provided by the healthcare system exchange.

8. The method of claim 1, wherein the receiving a set of communication rules comprises receiving a plurality of sets of communication rules;
wherein each of plurality of sets of communication rules correspond to different groups of healthcare systems, wherein each of the different groups comprise at least two healthcare systems; and
wherein the healthcare system exchange varies a communication protocol between different of the different groups of healthcare systems to ensure conformance to the set of communication rules corresponding to a group of healthcare systems.

9. The method of claim 1, wherein the request is generated automatically based upon a status of a patient within the first of the at least two healthcare systems.

10. The method of claim 1, wherein the resource comprises at least one of: a bed, a healthcare provider, a healthcare support staff, a procedure, and a piece of healthcare equipment.

11. A system, the system comprising:
a database, wherein the database comprises a set of communication rules corresponding to communications occurring between at least two healthcare systems, the set of communication rules defining information that can be shared between the at least two healthcare systems;
at least one network device facilitating communicative connection between a healthcare system exchange and the at least two healthcare systems;
a processor operatively coupled to the database and the at least one network device;
a memory device that stores instructions that, when executed by the processor, causes the information handling device to:
provide the healthcare system exchange, wherein the healthcare system exchange facilitates communicative connection of at least two healthcare systems based upon the set of communication rules corresponding to the at least two healthcare systems;
receive, at the healthcare system exchange from a first of the at least two healthcare systems, a request for access to a resource of at least a second of the at least two healthcare systems; and
transmit, from the healthcare system exchange to the at least a second of the at least two healthcare systems, the request, wherein the transmitting comprises ensuring the request conforms with the set of communication rules.

12. The system of claim 11, further comprising a graphical user interface associated with the healthcare system exchange.

13. The system of claim 12, wherein the graphical user interface displays at least one user input field for generating the request for access.

14. The system of claim 12, wherein the graphical user interface is provided within a system of at least one of the at least two healthcare systems and is updated and managed based upon instructions provided by the healthcare system exchange.

15. The system of claim 11, wherein the at least two healthcare systems comprise non-affiliated healthcare systems.

16. The system of claim 11, further comprising receiving, at the healthcare system exchange from the at least a second of the at least two healthcare systems, a status update related to the request; and
transmitting, from the healthcare system exchange to the first of the at least two healthcare systems, the status, wherein the transmitting comprises ensuring the status conforms with the set of communication rules.

17. The system of claim 16, wherein the status is displayed as at least one graphical element within a graphical user interface associated with the healthcare system exchange; and
wherein the at least one graphical element is iteratively updated based upon changes to the status as provided by the healthcare system exchange.

18. The system of claim 11, wherein the receiving a set of communication rules comprises receiving a plurality of sets of communication rules;
wherein each of plurality of sets of communication rules correspond to different groups of healthcare systems, wherein each of the different groups comprise at least two healthcare systems; and
wherein the healthcare system exchange varies a communication protocol between different of the different groups of healthcare systems to ensure conformance to the set of communication rules corresponding to a group of healthcare systems.

19. The system of claim 11, wherein the request is generated automatically based upon a status of a patient within the first of the at least two healthcare systems.

20. A product, the product comprising:
a computer-readable storage device that stores executable code that, when executed by a processor, causes the product to:
provide a healthcare system exchange, wherein the healthcare system exchange facilitates communicative connection of at least two healthcare systems;
receive, at the healthcare system exchange, and storing, within a database of the healthcare system exchange, a set of communication rules corresponding to communications occurring between the at least two healthcare systems, the set of communication rules defining information that can be shared between the at least two healthcare systems;
receive, at the healthcare system exchange from a first of the at least two healthcare systems, a request for access to a resource of at least a second of the at least two healthcare systems; and
transmit, from the healthcare system exchange to the at least a second of the at least two healthcare systems, the request, wherein the transmitting comprises ensuring the request conforms with the set of communication rules.
